(19) United States
(12) Patent Application Publication
(10) Pub. No.: US 2026/0345000 A1
(43) Pub. Date: Nov. 5, 2026

(54) SYSTEM AND METHOD FOR DIAGNOSIS AND TREATMENT OF VARIOUS MOVEMENT DISORDERS AND DISEASES OF THE EYE

(71) Applicant: CARMEL HAIFA UNIVERSITY ECONOMIC CORPORATION LTD., Haifa (IL)

(72) Inventors: Ilan SHIMSHONI, Haifa (IL); Ahmad KHATIB, Kfar Kana (IL); Shmuel RAZ, Kfar Vradim (IL)

(21) Appl. No.: 19/125,553

(22) PCT Filed: Oct. 31, 2023
(86) PCT No.: PCT/IL2023/051119

§ 371 Date: Apr. 29, 2025

Related U.S. Application Data
(60) Provisional application No. 63/420,949, filed on Oct. 31, 2022.

(51) Int. Cl. A61B 3/11; A61B 3/00; A61B 3/14; A61H 5/00

(52) U.S. Cl. CPC A61B 3/111; A61B 3/0025; A61B 3/145; A61H 5/005

(57) ABSTRACT

A system and method for diagnosis and treatment of at least one eye disorder is disclosed. The method may include, receiving from a camera a stream of images comprising the eyes of a user; identifying a location of a pupil center of each eye in each image; determining a distance between the pupils in each image; determining a distance between the camera the iris of each eye; calculating a near point of convergence (NPC) for the user, based on the distance between the pupils and the distance between the camera the iris of each eye; and diagnosing an eye disorder if the NPC is larger than a threshold value.

### CROSS-REFERENCE TO RELATED APPLICATIONS

This application claims the benefit of priority of U.S. Patent Application No. 63/420,949, filed Oct. 31, 2022, and titled: “SYSTEM AND METHOD FOR DIAGNOSIS AND TREATMENT OF VARIOUS MOVEMENT DISORDERS AND DISEASES OF THE EYE” which is hereby incorporated by reference in its entirety.

### FIELD OF THE INVENTION

The present invention relates generally to methods of diagnosing eye disorders. More specifically, the present invention relates to using a method for the diagnosis and treatment of various movement eye disorders using computer vision.

### BACKGROUND OF THE INVENTION

Convergence Insufficiency (CI) is characterized by a decreased ability to converge the eyes and maintain binocular fusion while focusing on a near target. In searching for the most common complaints of (CI), patients often complain of eye strain when reading, closing one eye when reading, or blurred vision after short periods of near work. The diagnosis of primary CI is based on the patient's presenting symptoms and the clinical signs. The etiology of isolated CI, not related to trauma or neurological disease, has not been completely determined. Although CI can present at almost any age, it is most common in the children and young adult population, and it is presented in 11-19 percent of children with an exodeviation. Exotropia is a form of strabismus (eye misalignment) in which one or both eyes turn outward. It is the opposite of crossed eyes, or esotropia. Exotropia may occur from time to time (intermittent exotropia) or may be constant and is found in every age group. The most common signs of CI include a large near point of convergence (NPC) and exodeviation near the NPC. Exodeviation occurs when the patient can no longer maintain fusion, this is referred to as the break point.

Currently, CI detection is done by a specialist (Usually an ophthalmologist), using a special ruler with a fixation target installed on it. The patient should fixate on the target, and by bringing the target closer to the patient's face, the distance from the patient's face at which fixation is lost is measured. This point is called the Near Point of Convergence (NPC). NPC is used to diagnose CI and to treat it.

CI treatment aims to reduce NPC. This is done by using the same principle of the diagnostic test. The patient performs a set of exercises to strengthen the eye muscles, by bringing a certain object closer to the patient's face to the point of fixation loss in a repetitive manner.

The test reliability depends on both the examiner's as well as the patient's performance. Several factors might affect the test results such as room illumination, examiner's distraction, and patient's cooperation being sometimes challenging or misleading, for example, in young children, mentally retarded patients, or even with normal adults. Moreover, the test is usually performed several times by the ophthalmologist to assure that the patient understands and performs well.

Convergence is the simultaneous inward movement of both eyes toward each other as can be seen in FIG. 1, to maintain a single binocular vision when viewing an object. Near Point of Convergence (NPC) Test: Near Point of Convergence test is a standard test for convergence ability. The needed equipment: A fixation target and a ruler as can be seen in (FIG. 2). The fixation target could be a pen or a pencil. To get the child's attention a tiny toy of a common character can be used, and it should be with small details that are clear to see. In (FIGS. 3,4) a special ruler is illustrated which is used for the convergence and accommodation test. In case of high-level myopia or hypermetropia or in case of presbyopia, the needed correction should be done in advance, prior to applying the test.

### Test Performing

In some embodiments, the way the test is performed may have great importance, and this directly affects the results. It therefore may be considered appropriate to perform the test.
(1) The Patient should sit down in a comfortable chair while looking directly at the fixation object that is placed at approximately 40-50 cm from the patient's nose.
(2) The room in which the test will be done should be well illuminated so the examiner could notice any minimal changes in eye movements.
(3) The examiner will start moving the fixation object slowly and smoothly close to the patient's nose.
(4) The patient will be instructed to pay attention, when the fixation object is seen double or very blurry. Also, the examiner should explain to the patient that the object can become a bit blurry, and that he should blink and still need to continue to fixate on it.
(5) While performing the test, the examiner should observe the patient's both eyes because some patients do not notice diplopia-when one of the eyes is not fixating to the object. As a result, this point is the near point of the convergence. In some cases, the patient was given instructions to ignore, what might be minor NPC, but still, the patient might report bit blurry as very blurry, since patients are bias.
(6) The test should be performed more than once to assure that the patient could understand and will perform the test properly. For the patients with convergence insufficiency repeated tests will show enlarging NPC.

### CI Treatment

As a consensus, orthoptic therapy is the primary treatment modality used by most eye care professionals for the treatment of CI. The plasticity of the fusional convergence reflex system allows patients to improve their convergence amplitudes with simple exercises. There are numerous different types of eye exercises; however, the primary treatment modalities for CI include home-based exercise, office-based visual therapy (OBVT) exercises, computer vergence exercises or a combination of these. Gradual Convergence Exercises (e.g., pencil pushups) are among the most famous treatments, which are performed by having the patient focus on a small target (usually an accommodative target) at a remote distance to acquire binocular single vision (BSV) and then slowly move the target toward their nose while maintaining BSV. A typical office-based visual therapy (OBVT) requires a patient to visit an optometrist's office or ophthalmologist's clinic for at least 1 hour every week for 12 weeks. They also claim that although OBVT with pencil push-ups for the home training has shown success rates as high as 73%, the home-based vision therapy often results in diminishing success rates down to just 33 percent over time. Thus, an alternative method is required to reduce outpatient visits by providing a reliable and effective home-based visual therapy. Surgical treatment is controversial and not routinely preformed.

### Strabismus

Strabismus is a condition in which the eyes do not properly align with each other when looking at an object. The condition may be present occasionally or constantly. Strabismus is considered a common disease, with a prevalence in the general population that can reach 5% while in children the prevalence is up to 7.1%. If present during a large part of childhood, it may result in amblyopia (also called lazy eye), which is a very important visual disorder that is caused by inability of eye-brain to work together effectively and it results in decreased vision in an eye that otherwise typically appears normal or loss of depth perception. If strabismus onset is during adulthood, it is more likely to result in double vision.

### Strabismus Detection Test—A State of Art Cover/Uncover Test

Diagnosis of strabismus by an ophthalmologist or specialist in the field is made as a gold standard by the “Cover/Uncover Test”. In this test the examiner asks the subject to look at a target, covers one eye at a time and observes the behavior of the other opened eye in those moments. The displacement of the eye in a certain direction can give the examiner an indication if subject has strabismus, as well as an assessment of the type of strabismus, and sometimes a rough assessment of the degree of strabismus depending on the examiner's experience. In order to perform an accurate calculation of the squint angle, a highlight experienced examiner needs to use an additional equipment called “Prism Bar”.

### MediaPipe Model and Eye Tracking

MediaPipe Model is a nonlimiting example, for an ML solutions for live and streaming media and the Eye Tracking, illustrated in FIG. 5, is an end-to-end neural network-based model that created a 3D mesh of 468 points represented on the human face. It relies on a single RGB camera input without the need for special sensors or additional instrumentation. This model detects the face in the original image, crops it as a rectangle (as illustrated in FIG. 6), which is provided as an input to the mesh prediction neural network, outputting vectors of 3D figures. In the training phase, they use a dataset of close to 30 k images taken by different phones, used in different capturing and lighting conditions. In addition, in order to obtain the structure of a real face, 3D morphable models (3DMM) were used. The final full model as shown in FIG. 6, detects and crops, as input, a face image in 256×256 size, then extracting a 64×64 feature map. At this point the division into submodels allows for obtaining 478 landmarks and creating a feature map of 24×24 specially for the eye area, from which the special area of the iris is extracted as a separate output. This allows for more free behavior of the iris regardless of other landmarks of the eye.

### SUMMARY OF THE INVENTION

Some aspects of the invention may be directed to a system and method for diagnosis and treatment of various eye disorders, The method may include, receiving from a camera a stream of images comprising the eyes of a user; identifying a temporal location of the iris and the pupil center of each eye in each image; determining a temporal distance between the pupils in each image; determining a distance between the camera and the iris of each eye; calculating a temporal near point of convergence (NPC) for the user, based on the temporal distance between the pupils and the distance between the camera the iris of each eye; and diagnosing an eye disorder if the NPC is larger than a first threshold value.

In some embodiments, the method further comprises identifying a movement of the eyelids, eyebrows, and changes in pupil size, and wherein diagnosing the eye disorder is based on the movement of the eyelids, eyebrows and changes in pupil size.

In some embodiments, the method further comprises: determining for each eye a temporal distance between the bridge of the nose and the pupil of each eye; and determining for each eye separately an eye disorder based on the temporal distances between the bridge of the nose and the pupil of each eye.

In some embodiments, the method further comprises: detecting changes in the distance between the pupils as function of the distance between the camera and the iris of each eye; and diagnosing the eye disorder based if the detected changes are larger than a second threshold value.\

Some additional aspects of the invention may directed to a method for diagnosis and treatment of strabismus comprising: (a) sending, via a user interface, instruction to a user to cover a first eye; (b) receiving from, a camera a first stream of images comprising the first and a second eye of a user; (c) identifying a location of the pupil of the second eye, in an image taken at the moment before a visual axis of the first eye is covered by the user; (d) determining a length of movement of the pupil of the second eye, in consecutive images; (e) repeating steps (a)-(d) when the second eye is instructed to be covered; and determining strabismus in at least one of the first eye and the second eye based on the length of movement of the pupil of each eye.

In some embodiments the method may further include repeating periodically steps (a)-(e); and estimating an effectiveness of a treatment provided to the user based on the length of movement of the pupil of each eye.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1, 2, 3, and 4 are images of NPC tests;

FIG. 5 is an illustration of MediaPipie Model architecture;

FIG. 6 is an illustration of facial mesh for MediaPipie Model;

FIG. 7A is a flowchart of a method of diagnosis and treatment of various eye disorders according to some embodiments of the invention;

FIG. 7B is an image and illustration of the focus of the eyes, showing key points on the eyelid (red) and iris (blue) including the center of the pupil according to some embodiments of the invention;

FIG. 7C is a graph showing raw measurements, taken from a healthy user, of the temporal distance between the pupils (PD) vs. the distance between the camera and the iris of each eye and the result of a smoothing algorithm, according to some embodiments of the invention;

FIGS. 7D and 7E are smoothened PD graphs of a healthy user (7D) in comparison to a user suffering from an eye disorder, according to some embodiments of the invention;

FIG. 7F shows smoothened graph showing the distance between each pupil and the nose median and the distance between the camera and the iris of each eye, according to some embodiments of the invention;

FIG. 8 is an illustration of the distance from eye to camera (d) calculated by using Focal Length (f) and the iris size according to some embodiments of the invention;

FIG. 9 is an illustration of Face Image Processing: a. Full face image with the cut line b. Image of the cut section which will be used later c. Face image after the smartphone becomes very close to the subject d. new face image after processing, according to some embodiments of the invention;

FIG. 10 includes images of faces before and after being processed according to some embodiments of the invention;

FIG. 11 is a graph dividing patients into Normal/CI based on an NPC threshold=13.41 cm according to some embodiments of the invention;

FIG. 12 is a flowchart of a method for diagnosis and treatment of strabismus, according to some embodiments of the invention;

FIG. 13 is an image showing a step in the method of FIG. 12, according to some embodiments of the invention; and

FIG. 14 is a block diagram, depicting a computing device which may be included in a system for diagnosis and treatment of various eye disorders according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

Unless explicitly stated, the method embodiments described herein are not constrained to a particular order or sequence. Additionally, some of the described method embodiments or elements thereof can occur or be performed simultaneously, at the same point in time, or concurrently.

Embodiments of the present invention disclose a method and a system for the diagnosis and treatment of various eye movement disorders. Such a system may diagnose patients who have CI-a disorder characterized by a decreased ability to converge the eyes and maintain binocular fusion while focusing on a near target, achieving that by using only the front camera of the smartphone with no need of additional equipment. The system and method use, are a face recognition platform, for example, MediaPipe Face Mesh, and a machine learning model for face and eye tracking, such as, Iris.

Some embodiments of the invention are directed to the development of an automatic CI detection and therapeutic application in smartphones, that could replace the typical test. The goal is to have an automatic system, for detection and treatment, that replaces the typical test procedure which has its own limitations: 1) The need for a professional tester. 2) In order to obtain high accuracy, special equipment is needed and is usually not available. 3) Collaboration of the subject: Ability to detect blurred vision or diplopia and report immediately to the examiner. 4) Patients with difficulty in verbal expression will not be able to undergo the test. 5) The above includes children and mental retardation.

Therefore, embodiments of the invention may include producing a simple smart system that requires minimum execution skill, and minimum cooperation from the patient, and gives results with high accuracy. The system is suitable for children. Patients can undergo the test by themselves without the need for the presence of an ophthalmologist at any time and as much as they want.

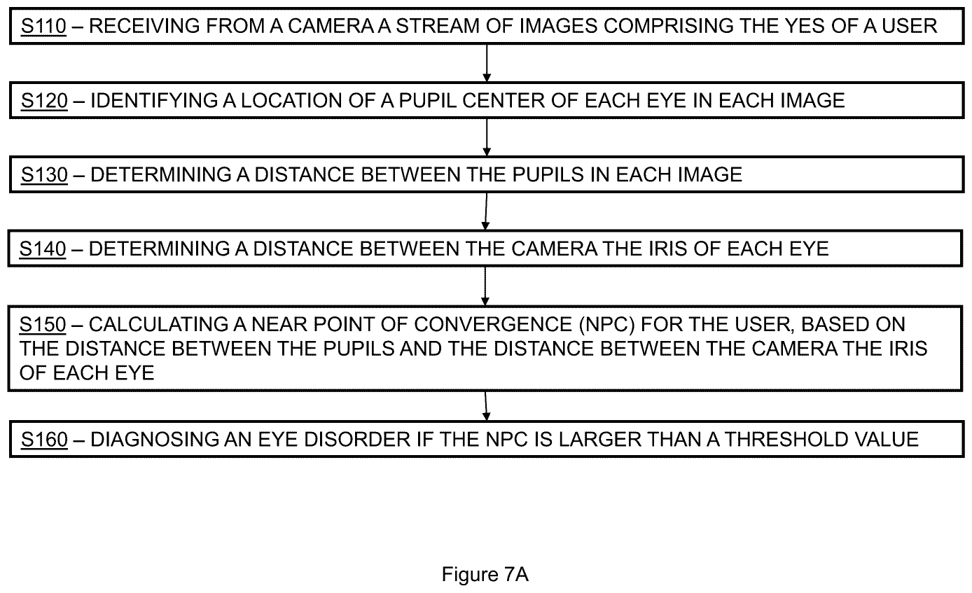

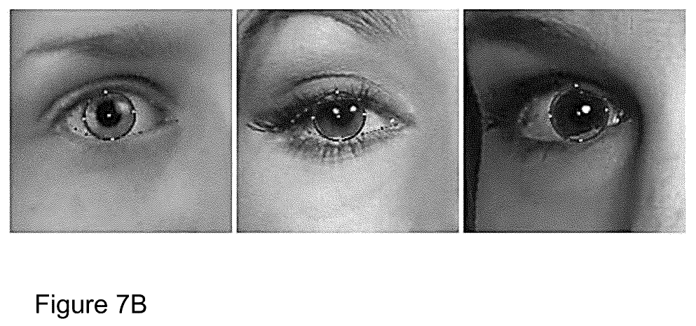

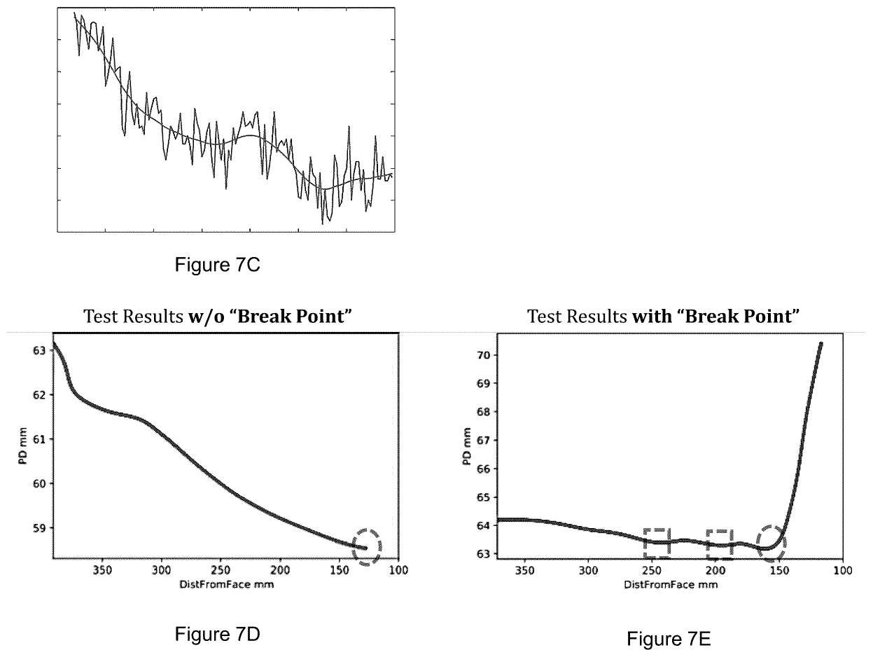

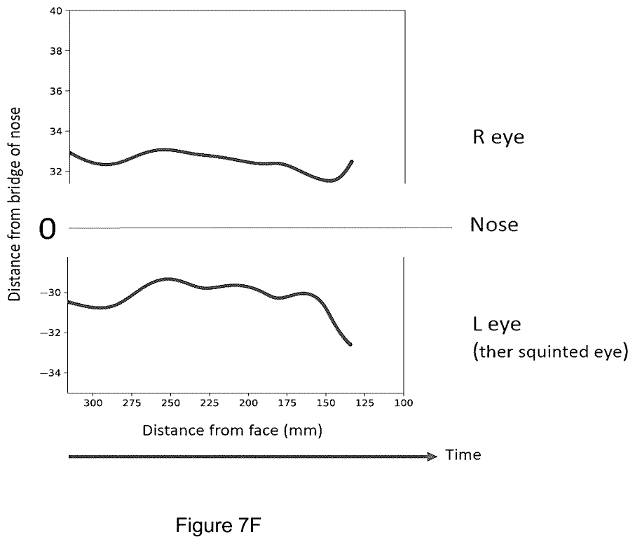

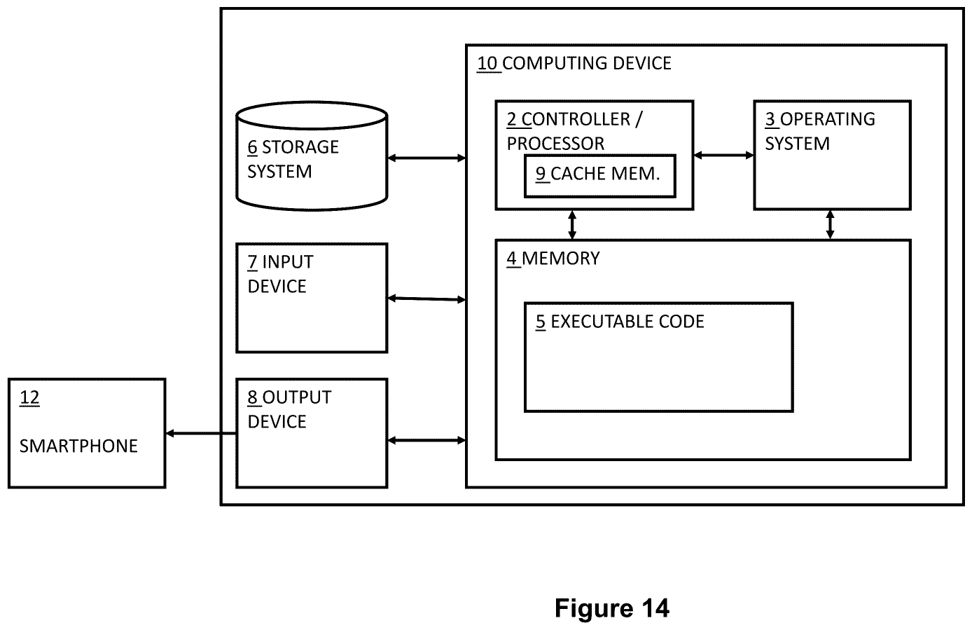

Reference is now made to FIG. 7A which is a flowchart of a method of diagnosis and treatment of various eye disorders according to some embodiments of the invention. The method of FIG. 7A may be performed by any computing device, for example, computing device 10 discussed and illustrated in FIG. 14. In step 110, a stream of images comprising the eyes of a user may be received from a camera, for example, the camera of a smartphone 12, illustrated in FIG. 14.

In step 120, a temporal location of the pupil center of each eye may be identified in each image. In some embodiments, the MediaPipe model may monitor eye movements in different situations, over time. For example, the model may detect the abnormal movement that the eyes make as soon as the fixation ability is lost (the “break point”) while performing the Near Point of Convergence (NPC) Test. By focusing on the eye area obtained from the grid of the face, which is the main output of the MediaPipe model, as can be seen in FIG. 7B, different behaviors of the eyes may be detected, for example, blinking and tracking the location of the eyes and the pupils at any given moment.

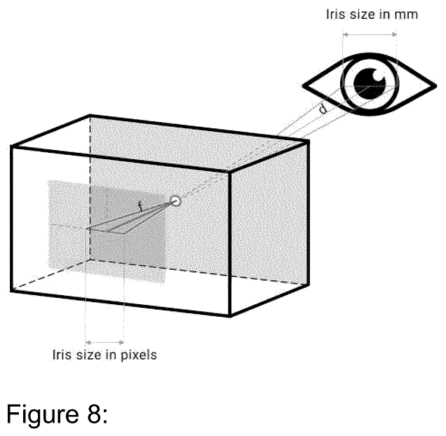

In step 130, a distance between the temporal pupils in each image may be determined, for example, by using the MediaPipe model which may also determine the temporal distance between the eyes and the pupils, which is also called the temporal Pupillary Distance (PD). In a nonlimiting example, MediaPipe may determine the location of the pupil center in each frame. This position, which is in 2D, and the coordinates of x and y obtained are the position in relation to the width or height of the image. And the use of image size in pixels gives the position x and y from the (0,0) point which is the corner of the image, and as a result the distance between the pupils is obtained in pixels. The fact that the size of the iris is relatively constant in the wide population (e.g., 11.7 mm, as explained herein) may be useful. One of these advantages may be the ability to convert the distance between the pupils from percent or pixels to millimeters in any given frame. The Pupillary Distance (PD) may be calculated using equation (1), as illustrated in FIG. 8.

$$PDmm = \frac{( RightEyeCenter . X - LeftEyeCenter . X ) * 11.7 mm}{IrisDiameter} , \qquad ( 1 )$$

where the “PD” is the pupillary distance in millimeters-which is the distance between the center of the eyes, the “RightEyeCenter.X” is the landmark's X component of the center of right iris in pixels, and the “LeftEyeCenter.X” is the landmark's X component of the center of left iris in pixels.

In step 140, a distance between the camera the iris of each eye may be determined. For example, Iris Related Depth Estimation is based on the fact that the size of the iris (in ophthalmology known as w-t-w white to white) is uniformly sized in a wide population with minimal differences. The estimated w-t-w is 11.7±0.5 mm. The method was tested for images received from several types of smartphones with good results and less than 10% error. The Iris-Camera distance (Distance-From-Camera (d)) in millimeters (mm) can be calculated without additional equipment, as seen in equation (2) and illustrated in FIG. 8.

$$DistanceFromCamera ( d ) = \frac{FocalLength ( f ) * 11.7 mm}{IrisDiameter} , \qquad ( 2 )$$

where the Distance-From-Camera (d) is the distance between the iris of one eye to the smartphone camera in millimeters, the Focal-Length (f) is the FL of the smartphone camera in pixels, and “Iris Diameter” is the diameter of the iris, of the same eye, in pixels.

The Focal Length in Pixels (FLp) by using front camera data while taking videos in 1×1 dimensions was calculated using equation (3).

$$FLp = \frac{FLmm}{( PS * \frac{W}{Wr} )} , \qquad ( 3 )$$

where the “FLp” is the camera focal length in pixels, the “FLmm” is the camera focal length in millimeters, the “PS” is the size of one pixel of the camera in millimeters, “W” is the width of the smartphone camera, which is the maximal width of an image that this camera can produce in pixels, and “Wr” is real width-which is the width of the frame's image that in pixels.

$$\frac{W}{Wr}$$

was used because the resulting image is smaller than the maximum resolution the camera can produce, and when the best resolution is produced then this value is equal to 1.

As known in the art, the original NPC test measures the distance between the center of the face-eye level- and the target to which the subject is staring at. Therefore, to be more accurate, and after calculating the distance of the camera from the eye, a system and method according to embodiments of the invention, performs another simple calculation, using similar triangles, and updates the distance to a central point between the eyes and not from the eye itself.

In some embodiments, the determined temporal PD may be plotted vs. the distance from camera (d) during the test, as illustrated in FIG. 7C as the two eyes are trying to focus on a target presented on the screen associated with the camera. As shown the raw data produced from the stream of images contains noise. Therefore, a smoothing algorithm was applied to the data resulting in a smooth graph. In the nonlimiting example of figured 7C, a Gaussian smoothing with a fixed sigma was performed, which resulted in a clear trend of progress and easy tracking of the change in PD in relation to the video's frame number and in relation to the distance from the face.

In step 150, the NPC for the user was calculated, based on the distance between the pupils and the distance between the camera the iris of each eye. A nonlimiting example is given below.

### A User Study

In some embodiments, in order to evaluate the method a user study was conducted to explore the technical and clinical potential of the mobile system. Excluded from the study: Patients with chronic eye diseases including history of eye surgery. Subjects wearing spectacles were included in our study.

Several short videos of the face were taken for each subject, while the subject was looking at the front camera as the smartphone approached the patient's face. A manual examination of all the frames was then performed, examining the level of accuracy of the mesh in the eye area and its ability to detect the iris. Second step: a manual and/or automatic measurement is performed, measuring the distance of the smartphone/camera from the center of the lower forehead and comparing it to the distance data that the system gave.

The system proved a high level of accuracy in identifying the iris circumference and the center of the pupil, but less accurate in identifying the eyelids contour. In the distance measurements, high accuracy was found in most frames.

Examining The System's Clinical Potential: To explore the clinical applications of the suggested system, we conducted a small user study which included twelve participants, with no history of chronic eye diseases or surgeries or current ophthalmic complaints. The study group included two women and ten men, age range 22 to 53 years (Table 1).

TABLE 1

| Method | P1 | P2 | | P3 | | P4 | | P5 | | P6 | P7 | P8 | P9 | P10 | P11 | | P12 | |
|---|---|---|---|---|---|---|---|---|---|---|---|---|---|---|---|---|---|---|
| Ophthalmologist | 18.5 cm | 25 | cm | 13 | cm | 15 | cm | 14.5 | cm | 12.5 cm | 12.5 cm | 11 cm | 17.5 cm | 15.5 cm | 10.5 | cm | 11 | cm |
| (Pencil Test) | | | | | | | | | | | | | | | | | | |
| Ophthalmologist | 12.5 cm | 30.5 | cm | 16.5 | cm | 21 | cm | 20 | cm | 10.5 cm | 13.5 cm | 13 cm | 17.5 cm | 14.5 cm | 13 | cm | 11.5 | cm |
| (Ruler Test) | | | | | | | | | | | | | | | | | | |
| Automatic system | 18.6 cm | 25 | cm | 17.9 | cm | 16.6 | cm | 15.1 | cm | 13.5 cm | 10.9 cm | 13 cm | 16.1 cm | 10.7 cm | 9.9 | cm | 12.2 | cm |

An ophthalmologist was present during the study to validate the results. The study was conducted as follows: at first, the ophthalmologist performed the near point of convergence test (NPC) on the participants in two different methods and in the following order: a pencil test, a thirty-minute rest, and then the ruler test. The near convergence points were computed. These points were considered the ground truth reference points. Then the participants were instructed to hold the smartphone in both hands while looking towards a small black dot on a white background located at the top of the screen and bring the phone closer to the face up to 0 distance. This video is used to calculate the automatic NPC in our system. Finally, the average of all the points above was computed and compared to evaluate the efficiency of the system.

The preliminary results show that a system and method according to embodiments of the invention may detect an NPC point with great success, with a statistically significant resemblance to a ground truth diagnostic test performed by an ophthalmologist, and this is reflected in the high detection ability of CI disease.

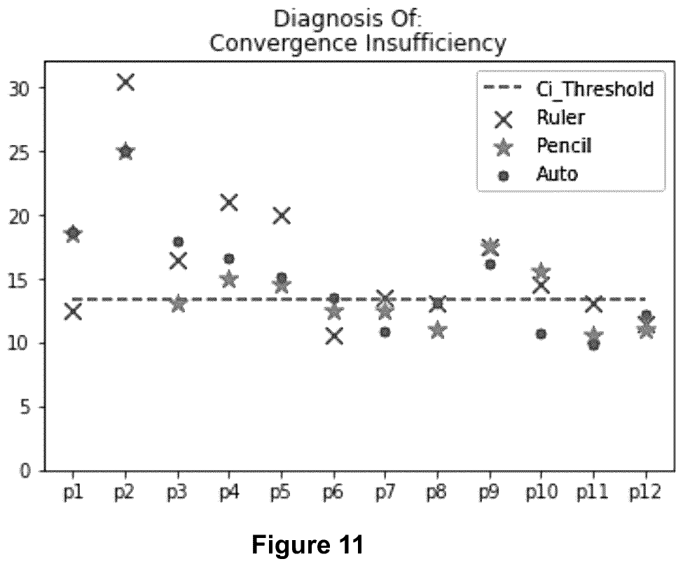

In step 160, an eye disorder is diagnosed if the NPC is larger than the first threshold value. For example, correlation tests showed that NPC detection according to embodiments of the invention is highly and significantly correlated with the Ruler, and medium-high correlated with the Pencil. Interestingly, the Pencil and the Ruler didn't show a high correlation. FIG. 11 shows that our method is eligible for CI detection, assuming that the upper value of the normal NPC point in the general population is 13.41 cm (e.g., the first threshold value). Shifting the focus to the comparison between the automated system and the human systems, a big match can be seen apart from patient number 10, which is the only time the system is alone on one of the two sides of the threshold. Only in two subjects (number 1 and 3) there was a mismatch between the manual and the automatic tests, suggesting excellent preliminary results.

In some embodiments, the PD may be plotted vs. the distance of the iris of each eye from the camera, in order to provide additional data assisting in diagnosing eye disorder. A comparison between two PD plots of a healthy person and a person with an eye disorder is shown in FIGS. 7D and 7E. Near Point of Convergence (NPC) or the “Break Point” is the point of maximal convergence, which is the point of minimal PD as well. As shown in FIG. 7C, the behavior of the healthy eyes during focusing shows a clear single NPC “breaking point” (which can be set as the first threshold value) at which the eyes can no longer be focused, at the end of the focusing process. While in the unhealthy eyes several minor NPCs “breaking points” can be detected prior to the major final breaking points. These minor breaking points can be identified by small changes (e.g., the first derivative) of in PD as a function of the distance between the camera and the iris of each eye. These small changes can set a second threshold value for diagnosing the eye disorder.

Accordingly, the method may include detecting changes in the PD as a function of the distance between the camera and the iris of each eye; and diagnosing the eye disorder based if the detected changes are larger than a second threshold value. In some embodiments, the detection of these changes may also prevent a false negative determination of the real NPC of the user, as the first NPC “breaking point” is at a larger distance than the final “breaking point”. These early minor “braking points” are impossible to be detected in the traditional human-conducted tests.

In some embodiments, detecting a movement of the eyelids, and eyebrows, and changes in pupil size may provide additional data that may allow diagnosing the eye disorder based also on the movement of the eyelids, and eyebrows and changes in pupil size. These movements may also be included in the AI algorithm and may improve the prediction accuracy of the AI algorithm.

Reference is now made to FIG. 7F which shows the distance between each pupil and the nose median plotted against the distance between the camera and the iris of each eye, during focusing of the eyes, according to some embodiments of the invention. As shown in FIG. 7F each eye has different focusing behavior. In the nonlimiting example of FIG. 7F the right eye shows a smooth and normal behavior with one distinctive breaking point, while the left eye shows several minor breaking points, the first one already at 250 mm. As shown each eye has a number of minor NPCs, points that can vary in number and location from eye to eye. In some embodiments, each eye has one Major NPC, which can be different in its location from the other eye and from the major NPC of both eyes together, which is actually, the final result of the state of art NPC test.

Such a diagnosing method may allow diagnosing the “weak” or unhealthy eye, and therefore providing more suitable treatment for the unhealthy eye. For example, instead of providing focusing exercises for both eyes, only the unhealthy eye will be exercised to strengthen convergence or accommodation of the eye. In yet another example, vision tests may be conducted on each eye, and correcting glasses may be provided to the unhealthy eye. Lazy eye treatments associated with weak convergence ability may be performed, including refractive correction (regardless of age or presence of presbyopia), patch therapy, atropine therapy. Therefore, additionally or alternatively, the method may include determining for each eye a temporal distance between the bridge of the nose and the pupil of each eye and determining for each eye separately an eye disorder based on the temporal distances between the bridge of the nose and the pupil of each eye.

### Applications

Convergence Insufficiency: Application for assessing convergence insufficiency—At first the ophthalmologist performed the near point of convergence test (NPC) three times on the participants, and the near convergence points will be computed. These points will be used as ground truth reference points. Then the participants will be instructed to use the mobile app, in this case the near convergence points were computed automatically, three times. Finally, the average of the computed near convergence points in two tests, manually by the ophthalmologist and automatically by the mobile app are compared to evaluate the efficiency of the system. Machine learning methodologies will be applied using the ophthalmologist ground truth for the training of the machine.

Therapeutic application for convergence insufficiency—The therapeutic system relies on the principle of practice, the goal of which is to strengthen the patient's convergence ability by gradually training the patient's visual system which results in a lower NPC value. Our system gives the patient the option to build a training program that suits his lifestyle: the number of trainings per day (2 or 3 trainings), the number of repetitions of the exercise in each training set (5, 10 or 15 repetitions) and the duration of the training period (1, 2 or 3 months). At the end of each training set, the system displays the NPC values obtained in each exercise, the average of the results, the percentage of improvement from the training set he did, and a graph describing the number of training set versus the average of the measurements in that practice. In addition, the system keeps all the information received from the treatment system, giving a reminder to the patient before any training set. Finally, the system transmits to the attending ophthalmologist, once a week, the results of the training sets, with the appropriate graphs including comparison and the patient's progress rate, and by relying on these data the attending ophthalmologist will be able to recommend a change in treatment plan.

Application for assessing Strabismus—The diagnosis will be based on iris-pupil tracking of each eye separately from the other eye obtained from the video captured by selfie camera of the smartphone, and it is all based on the principle that both eyes work in full synergism including the motility system, disruption in this synergism will lead to medical problems such as strabismus, which is a large package of eye diseases most of which are treatable. Another important issue for the diagnosis that our system makes is the matter of early detection of those diseases which can prevent lazy eye (Amblyopia), one of the serious ophthalmology problems, that because after a certain age there are no longer proven treatments for this disorder, neither in literature nor in practice.

Help with the Lazy Eye (Amblyopia) Treatment Process:

As mentioned above, the main treatment for a lazy eye that caused by strabismus, is closure of the good eye, with or without glasses depending on the patient's condition, in this way the weak eye is forced to work and take on more role in the vision process. Non treating of amblyopic eye can lead to severe vision damage, irreversible, can lead to blindness in difficult situations. The treatment plan, which includes a decision on which eye to close, the time of closing a day, the duration of the closing plan, is determined by a single examination of the patient by an ophthalmologist every few months (sometimes half a year or more).

A home self-examination of the patient in the system, according to a known schedule, can give a very important indication of the effectiveness of the treatment plan, the need to change the treatment plan metrics before visiting the attending ophthalmologist, which will be very helpful in treatment and give the ophthalmologist a much broader view over the patient medical condition.

### Additional Diagnostic Products

Application for assessing “Accommodation Range”: The same ruler which used to check the NPC point may be used to determine the “Accommodation Range”, which basically reflects the range in which the subject has the ability to read lines from a distance close to the face in a sharp and clear manner. The system may have the ability to detect changes in accommodation based on changes in eye movements and pupil size and other signs that may indicate loss of accommodation or being into a state of accommodation, which has its own clinical reflections. This capability enriches the benefits of our system and strengthens our belief that our research will further contribute to science and medicine.

Application for assessing color vision of the user: The system may give an assessment of the color vision of the patient by a short test-exposing the patient to various color images that will appear in front of him on the phone screen, without the need for user response, but evaluates the change in convergence-accommodation in response to a change in colors. The system relies on two well-known principles in the literature: the first is the change in accommodation in response to a change in colors, and that the change in convergence and accommodation requires identification of the object to which one is looking. The system is important for any user who wants to know the status of his color vision in a fast, smart, simplified way, but it is especially important in its ability to test color vision in the speechless or impaired communication skills, in children, in patients with mental retardation and certain cognitive condition and others.

### Tuning the System and Method

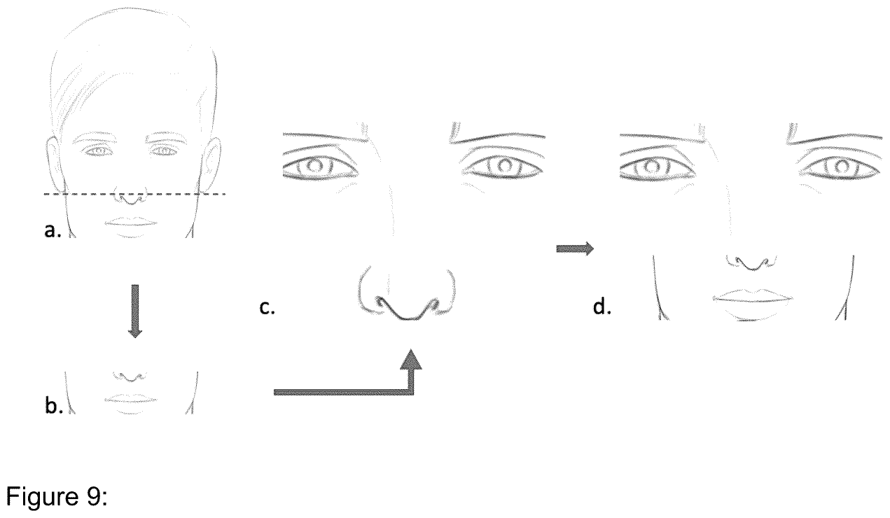

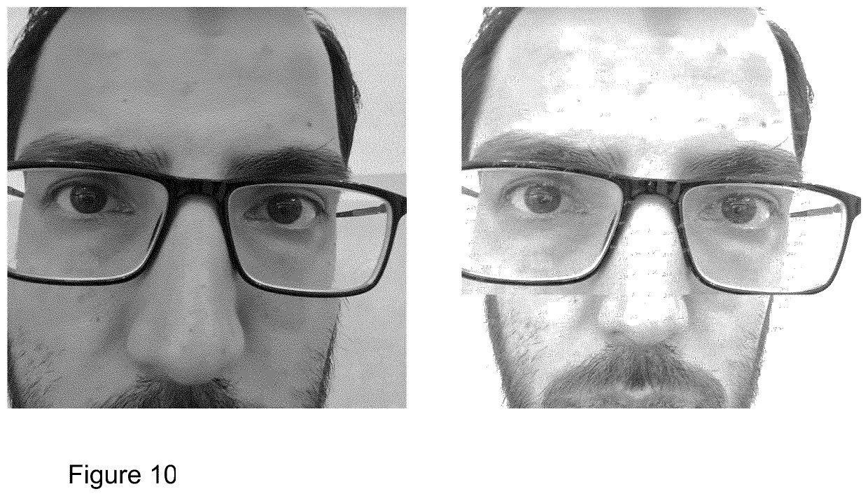

In some embodiments, during testing the above formula for calculating the distance from the camera, one of the MediaPipe algorithm limitations were encountered, the inability to detect the face and the eyes from very close distances. Therefore, during testing it was difficult to follow the eyes from distances less than 10-12 cm. Therefore, for example, if the eyes did converge down to 11 cm distance, then the NPC is less than 11 cm from the face. To solve this limitation the following steps were conducted.
(1) The landmark indicating the tip of the nose was followed, as soon as this point became close to about 35 pixels from the lower border of the image as illustrated in FIG. 9-a, this area was cut from the image and set aside for future use in the next step as shown in FIG. 9-b.
(2) As soon as the algorithm fails to recognize the landmarks from the face image, the process was stopped that problematic image: paste the patch image cut earlier on the bottom of this image and finally get a new processed image as illustrated in FIG. 9-d and can be seen in real image in FIG. 10, and then feedback the new image to the MediaPipe algorithm.
(3) The data and results were stored until the algorithm failed to process new images and this is considered the end of the processing work.

The above steps allow the algorithm to keep processing while the smartphone continues its progress towards the subject's face for another 1 to 2 cm, meaning that approaching a distance of 10 cm from the face and in some subjects even less than 10 cm is applicable. In the literature an NPC point below 10 cm is considered normal.

Machine learning tools were exploited to see the sensitivity and specificity of our approach compared with the ruler and the pencil ground truths, using the NPC threshold=13.41 cm, Ground Truth=Ruler. The sensitivity of our methods with the pencil as ground truth is 83.33% and the specificity is 66.67% with overall accuracy of 75%. The sensitivity of our methods with the ruler as ground truth is 71.42% and the specificity is 60% with overall accuracy of 66.6765.71%.

### Diagnosis and Treatment of Strabismus

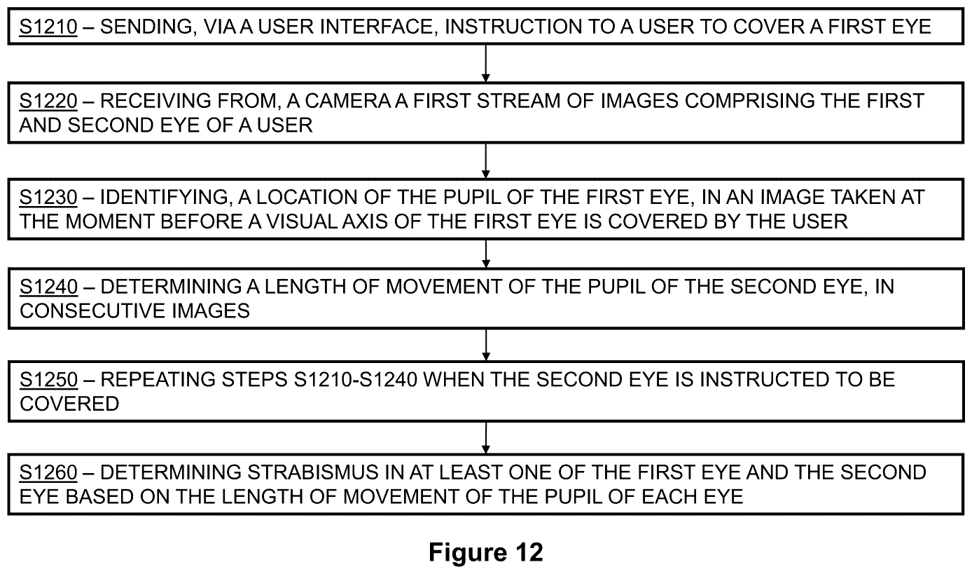

Reference is now made to FIG. 12 which is a flowchart of a method for diagnosis and treatment of strabismus according to some embodiments of the invention. The method of FIG. 12 may be performed by a similar system performing the method of FIG. 7A (e.g., computing device 10 of FIG. 14) or by any other suitable system. In step 1210, a user may receive, from a user interface, instructions to cover his/her first eye (e.g., by his/her hand). For example, the user may receive from his/her smartphone 12 audio messages, illustrated in FIG. 14, see on the screen visual instructions of literal instructions for covering the left eye.

In step 1220, a first stream of images comprising the first and second eye of a user is received from a camera, for example, the camera of smartphone 12.

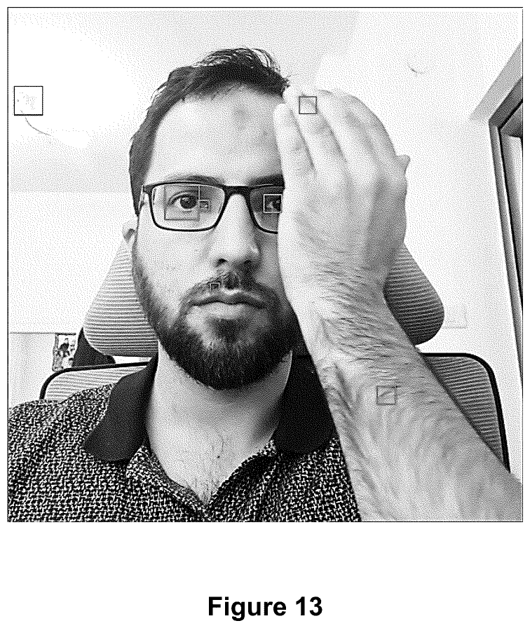

In step 1230, a location of the pupil of the first eye, may be identified in an image taken at a moment before a visual axis of the first eye is covered by the user. Such an image is illustrated in FIG. 13, where the location of the pupil of the right eye is detected at a moment before a visual axis of the right eye is covered. For example, the controller may use similar methods to the ones discussed with respect to step 120.

In step 1240, a length of movement of the pupil of the second eye is determined from consecutive images. Computing device 10 may recognize in each consecutive image following the image taken at a moment before a visual axis of the first eye is covered by the user, the location of the pupil of the first eye. Computing device 10 may then calculate the total length the pupil moved from the initial location at the image taken at a moment before the visual axis of the first eye is covered by the user, to the final location at which the pupil stops moving.

In some embodiments, the temporal location of the pupil of the first eye may be plotted as a function of time or image number (e.g., frame number) and a smoothing algorithm may be conducted (e.g., as discussed herein above) in other to filter undesired noise from the detection.

In step 1250, steps 1210-1240 may be repeated for the second eye.

In step 1260, strabismus is determined in at least one of the first eye and the second eye based on the length of movement of the pupil of each eye. For example, strabismus is determined if the length of movement of the pupil of at least one eye is greater than a threshold value.

In some embodiments, the method may include performing an Automated Bielschowsky three-step test, for calculating a rotation degree of the face. In some embodiments, the method may have the ability to diagnose almost all types of strabismus. The method may further be able to decide which is the squinting eye, which muscle is problematic, and also if it is a muscle weakness or restriction, and to give a good estimate of the degree of strabismus.

In some embodiments, the system and method may allow self-diagnosis without the need for the presence of an expert in the field during the test, due to minimal actions required of the patient such as holding the smartphone in front of the face. An application running on smartphone 12 and/or computing device 10 may be able to differentiate between permanent strabismus; “Tropia”, and non-permanent strabismus; “Phoria”, and “Intermittent Strabismus”, if it is “Horizontal” or “Vertical” or “oblique” strabismus, deciding which is the squinting eye, which muscle is problematic, and also if it is a muscle weakness or restriction and give an estimation of the strabismus's angle.

In some embodiments, the system may provide a diagnosis of the medical problem and an initial recommendation to the patient for the next step in diagnosis or treatment, as well as our system will be able to interface with other systems of telemedicine. The diagnosis may be based on iris-pupil tracking of each eye separately which obtained from the video captured by “selfie” camera of the smartphone, and it is all based on the principle that both eyes work in full synergism including the motility system, while disruption in this synergism will lead to medical problems such as strabismus, which is a large package of eye diseases most of which are treatable. The system and method may allow an early detection of diseases which can prevent lazy eye (Amblyopia), one of the serious ophthalmology problems, that because after certain age there are no longer proven treatments for this disorder, neither in literature nor in practice.

In some embodiments, “Latent Strabismus” may be detected. Latent Strabismus is a group of strabismus, which appear at certain conditions and not regularly. The theory is that impaired binocular vision (for various reasons) will result in the inability of the brain and eyes to maintain a single image in the subject's visual field and this is manifested in strabismus. We will improve the system's capabilities so that it can work continuously and for a long time, it can help diagnose the above types of strabismus, and give a lot of information for the characterization of this strabismus, information that an ophthalmologist will not be able to get in one or more tests at his clinic. For example: During prolonged work of the patient in front of screens or prolonged reading, the system can detect the moment when the patient experiences the first strabismus event. In addition, to count the strabismus events that have been in the given time range, which can give an indication of the time the patient is reading and working in one eye without his notice, which has many implications for the quality of work or learning of the subject. The above data can greatly help the ophthalmologist in assessing the patient's medical status and prescribing him a treatment plan and follow-up accordingly and even monitoring the effectiveness of the various treatments including surgical one.

In some embodiments, the method may further include repeating periodically steps 1210-1240 and estimating effectiveness of a treatment provided to the user based on the length of movement of the pupil of each eye. For example, the main treatment for a lazy eye that is caused by strabismus, is closure of the good eye, with or without glasses depending on the patient's condition, in this way the weak eye is forced to work and take on more role in the vision process. No treatment of amblyopic eye can lead to severe vision damage, irreversible, which can lead to blindness in difficult situations. The treatment plan, which includes a decision on which eye to close, the time needs for closing a day, the duration of the closing plan, all that is determined, may be periodically tested and updated according to the progress of the user.

A method according to embodiments of the invention may include a home self-examination of the patient, at to a known schedule. This method may provide a very important indication of the effectiveness of the treatment plan, the need to change the treatment plan metrics before visiting the attending ophthalmologist, which will be very helpful in treatment and give the ophthalmologist a much broader view over the patient medical status.

### Accommodation Range

Some additional embodiments of the invention may be directed to an automatic accommodation range detection. As known in the art, the same ruler which used to check the (NPC, illustrated in FIG. 3) is used to determine the “Accommodation Range”, which basically reflects the range in which the subject has the ability to read lines from a distance close to the face in a sharp and clear manner. The system according to embodiments of the invention has the ability to detect changes in accommodation based on changes in eye movements and pupil size and other signs that may indicate loss of accommodation or being into state of accommodation, which has its own clinical reflections. This capability enriches the benefits of our system and strengthens our belief that our system will further contribute to science and medicine.

In some embodiments, the algorithm disclosed herein above may be used while presenting to the user a line with a plurality (e.g., 4-6) letter or symbols, lowing the user to identify the letters or symbols during the test. In such case the algorithm may calculate the point at which the user loses focus, and the point of return to focus. The algorithm may use these calculation to determine accommodation range.

Reference is now made to FIG. 14, which is a block diagram depicting a computing device, which may be included within an embodiment of a system for diagnosis and treatment of various eye disorders, according to some embodiments.

Computing device 10 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 10 may be included in, and one or more computing devices 10 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 10, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task or script. Executable code 5 may be executed by processor or controller 2 possibly under control of operating system 3. For example, executable code 5 may be an application that may diagnose, and treat of various eye disorders as further described herein. Although, for the sake of clarity, a single item of executable code 5 is shown in FIG. 14, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Previously recorded NPCs may be stored in storage system 6 and may be loaded from storage system 6 into memory 4 where it may be processed by processor or controller 2. In some embodiments, some of the components shown in Figurel may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 10 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 10 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

A neural network, e.g. a neural network implementing machine learning, may refer to an information processing paradigm that may include nodes, referred to as neurons, organized into layers, with links between the neurons. The links may transfer signals between neurons and may be associated with weights. A NN may be configured or trained for a specific task, e.g., pattern recognition or classification. Training a NN for the specific task may involve adjusting these weights based on examples. Each neuron of an intermediate or last layer may receive an input signal, e.g., a weighted sum of output signals from other neurons, and may process the input signal using a linear or nonlinear function (e.g., an activation function). The results of the input and intermediate layers may be transferred to other neurons and the results of the output layer may be provided as the output of the NN. Typically, the neurons and links within a NN are represented by mathematical constructs, such as activation functions and matrices of data elements and weights. A processor, e.g., CPUs or graphics processing units (GPUs), or a dedicated hardware device may perform the relevant calculations.

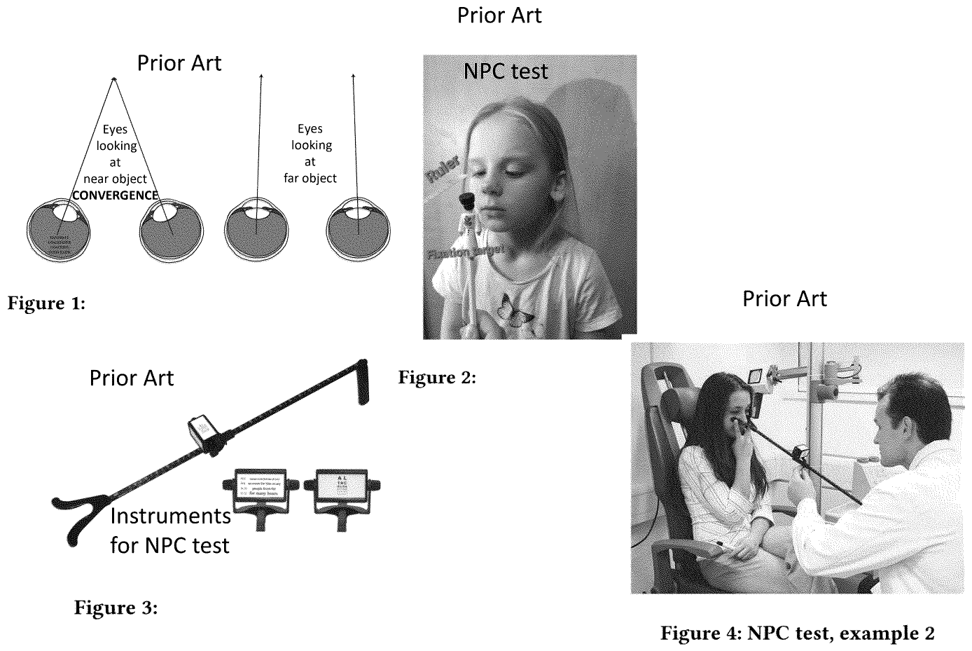

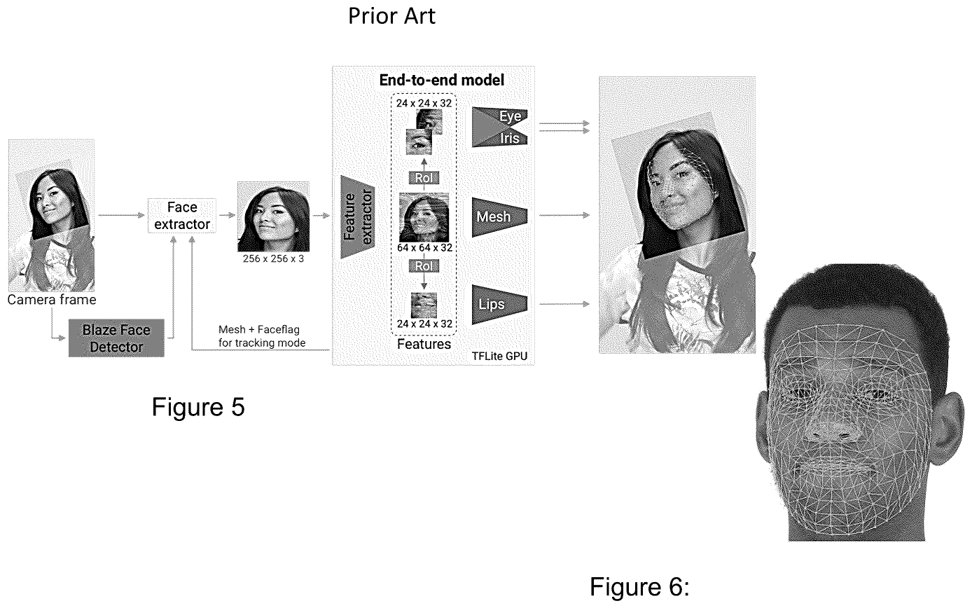

According to some embodiments of the invention, a system may be implemented as a software module, a hardware module or any combination thereof. For example, system may be or may include a computing device such as element 1 of FIG. 1, and may be adapted to execute one or more modules of executable code (e.g., element 5 of FIG. 14) to diagnose and treat various eye disorders, as further described herein.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A method for diagnosis and treatment of at least one eye disorder, comprising:
receiving from a camera a stream of images comprising the eyes of a user;
identifying a temporal location of the iris and the pupil center of each eye in each image;
determining a temporal distance between the pupils in each image;
determining a distance between the camera and the iris of each eye, calculating a temporal near point of convergence (NPC) for the user, based on the distance between the pupils and the distance between the camera and the iris of each eye; and
diagnosing an eye disorder if the temporal NPC is larger than a first threshold value.

2. The method of claim 1, wherein the at least eye disorder is Convergence Insufficiency (CI).

3. The method of claim 1, further comprising identifying a movement of the eyelids, a movement of the eyebrows, and changes in pupil size, and wherein diagnosing the eye disorder is based on at least one of, the movement of the eyelids, the movement of the eyebrows and changes in pupil size.

4. The method of claim 1, further comprising:
determining for each eye a temporal distance between the bridge of the nose and the pupil of each eye; and
determining for each eye separately an eye disorder based on the temporal distances between the bridge of the nose and the pupil of each eye.

5. The method according to claim 1, further comprising:
detecting changes in the distance between the pupils as function of the distance between the camera and the iris of each eye; and
diagnosing the eye disorder if the detected changes are larger than a second threshold value.

6. A method for diagnosis and treatment of strabismus, comprising:
(a) sending, via a user interface, instruction to a user to cover a first eye;
(b) receiving from, a camera a first stream of images comprising the first and a second eye of a user;
(c) identifying, a location of the pupil of the second eye, in an image taken at the moment before a visual axis of the first eye is covered by the user;
(d) determining a length of movement of the pupil of the second eye, in consecutive images;
(e) repeating steps (a)-(d) when the second eye is instructed to be covered;
(f) determining strabismus in at least one of the first eye and the second eye based on the length of movement of the pupil of each eye.

7. The method of claim 6, further comprising:
repeating periodically steps (a)-(e); and
estimating an effectiveness of a treatment provided to the user based on the length of movement of the pupil of each eye.
